(19) United States
(12) Patent Application Publication
(10) Pub. No.: US 2021/0154210 A1
(43) Pub. Date: May 27, 2021

(54) Ophthalmic Formulations and Uses Thereof

(71) Applicants: Iskender Alkin Solmaz, Istanbul (TR); Isilay Kavadarli, Istanbul (TR); Canan Sevimli Gur, Izmir (TR); Murat Kubilay Turkmen, Burlington (CA)

(72) Inventors: Iskender Alkin Solmaz, Istanbul (TR); Isilay Kavadarli, Istanbul (TR); Canan Sevimli Gur, Izmir (TR); Murat Kubilay Turkmen, Burlington (CA)

(21) Appl. No.: 17/163,119

(22) Filed: Jan. 29, 2021

Related U.S. Application Data
(63) Continuation of application No. PCT/US2019/042368, filed on Jul. 18, 2019.

(30) Foreign Application Priority Data
Jul. 31, 2018 (TR) 201811088

(51) Int. Cl. A61K 31/58; A61K 47/18; A61K 47/54; A61K 47/02; A61K 47/38; A61K 47/32; A61K 47/10; A61K 47/20; A61K 9/00; A61P 27/02

(52) U.S. Cl. CPC A61K 31/58; A61K 47/186; A61K 47/547; A61K 47/02; A61P 27/02; A61K 47/32; A61K 47/10; A61K 47/20; A61K 9/0048; A61K 47/38

(57) ABSTRACT

Provided herein are ophthalmic formulations and eye drops that contain a telomerase activator as the active agent to increase telomerase activity. Also provided are methods for reducing the incidence of age-related eye conditions or for increasing viability of cells in the corneal tissue by administering or contacting the ophthalmic formulation to one or both eyes in a subject.

### CROSS-REFERENCE TO RELATED APPLICATIONS

This application is a continuation under 35 U.S.C § 120 of pending international patent application PCT/US2019/042368, filed Jul. 18, 2019, which claims benefit of priority under 35 U.S.C. § 119(a) of Turkish Application No. 2018/11088, filed Jul. 31, 2018, the entirety of which is hereby incorporated by reference.

### BACKGROUND OF THE INVENTION

### Field of the Invention

The invention relates to ophthalmic formulations and eye health. More specifically the present invention is directed to ophthalmic formulations for activating telomerase and methods for increasing the viability of corneal tissues or cells by activating telomerase.

### Description of the Related Art

The eye is one of the first sensory organs to experience the signs of aging. While natural age-related changes may have direct impact on vision, age related illnesses unrelated with the eye can also impact the eye. Astigmatism, myopia, hypermetropia, presbyopia, hordeolum, glaucoma, amblyopia, cataract, diplopia, nyctalopia, eye pain, red eye, eye allergies, xerophthalmia, tearing, swollen eyelid and age-related macular degeneration are common ophthalmic diseases. There are several eye diseases that start without any early symptoms. For example, the pupil gets smaller and its response to light weakens with age leading to difficulties in seeing in dim light and delayed dark-light adaptation. Flexibility of the crystalline lens also diminishes with age resulting in presbyopia. Aging also affects the fibers attached to the ciliary body, leading to impaired refractive capacity. The transparent eye lens also starts to turn yellow with age, reducing transmission, increasing the amount of blue light absorbed, and increasing scattering thereby impairing color perception and contrast. Dry eye (keratoconjunctivitis sicca) is another ailment caused by aging associated tear hyperosmolarity and epithelial cell damage leading to aqueous layer failure.

The most important reason for central vision loss in Americans over the age of 50 is age related macular degeneration. Prevalence of this disease is roughly 85 to 90% for dry type and 10 to 15% for neovascular type. Aging related changes on the macula, which affects the outer retina, the retina pigment epithelium (RPE), the Bruch membrane and choriocapillaries cannot always be diagnosed clinically. Glaucoma is another eye disease that is characterized with high levels of intraocular pressure that impairs the optic nerve head. The two primary types of glaucoma are congenital and acquired glaucoma. Approximately 2% of people over the age of 40 and 10% over the age of 80 suffer from acquired glaucoma.

The telomere is a special heterochromatin structure present on the edges of eukaryotic chromosomes. Telomeres might have a length from 1-50 kb depending on type of cell and genetic history. Human telomeres are 10-15 kb in reproductive cells but are shorter in some somatic cells. Telomeres protect chromosome edges from random double chain DNA fragmentation thereby avoiding fusion of chromosomes. Besides this physical protection of chromosomes, eukaryotic telomeres have important cellular functions such as chromatin organization, chromosome replication and cell reproduction. Telomere length in DNA is reduced by about 50 to 150 base pairs for each cell division and this shortening eventually restricts the number of cell divisions a cell can make leading to accelerated or delayed control of cell aging.

The most common method for maintaining telomere length is increasing telomerase activity. The ability of telomerase to protect cell demise has led to an interest in developing telomerase inhibitors to treat cancer. In contrast, telomerase activators are expected to slow down the aging process and hence may be relevant to the treatment of aging-related eye diseases. While there have been some efforts to increase telomerase activity by delivering telomerase into cells, the art is deficient in formulations that may be used to slow the aging process to treat eye conditions. The present invention fulfills this longstanding need and desire in the art.

### SUMMARY OF THE INVENTION

The present invention is directed to an ophthalmic formulation. The ophthalmic formulation comprises, in an isotonic solution, a telomerase activator, an antimicrobial agent, a chelator and at least one excipient.

The present invention also is directed to an eye drops formulation. The eye drops formulation comprises cycloastragenol at a concentration of about 3 ng/ml to about 500 ng/ml in an isotonic saline solution containing an antimicrobial agent, a chelator and an excipient.

The present invention is directed further to a method for reducing the incidence of an age-related eye condition in a subject. In the method an amount of the ophthalmic formulation described herein that is effective to increase an activity of telomerase in the corneal tissue is administered to one or both eyes of the subject thereby reducing the incidence of age-related eye diseases.

The present invention is directed further still to a method for increasing viability of cells in the corneal tissue of a subject. In the method both eyes of the subject are contacted with an amount of the ophthalmic formulation described herein.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1B show an in vitro viability analysis in corneal endothelial cells. FIG. 1A shows a bar chart representation of cell viability in corneal endothelial cells treated with the indicated concentrations of Formulation eye drop formulation for 48 hours. FIG. 1B shows a Tendency/Regression Line and Tendency Equation of the cell viability data shown in FIG. 1A. IC₅₀ value was calculated from this graph to be 40.2 μg/mL.

FIGS. 2A-2B shows graphical representation and photomicrographs for the effect of Formulation eye drop formulation containing various concentrations of the active compound cycloastragenol in 1% DMSO, on viability of corneal endothelial cells. FIG. 2A is a cell viability analysis in corneal endothelial cells treated with the indicated concentrations of cycloastragenol in 1% DMSO. Data were analyzed 48 hours after treatment. FIG. 2B shows representative photomicrographs for each of the cycloastragenol concentrations in FIG. 2A.

FIGS. 3A-3B shows a graphical representation and photomicrographs of the effect of the Formulation eye drop formulation containing various concentrations of the active compound cycloastragenol in 0.2% DMSO, on viability of corneal endothelial cells. FIG. 3A is a cell viability analysis in corneal endothelial cells treated with the indicated concentrations of cycloastragenol in 1% DMSO. Data were analyzed 48 hours after treatment. FIG. 3B shows representative photomicrographs for each of the cycloastragenol concentrations in FIG. 3A

FIGS. 4A-4B show in vitro telomerase activity in corneal endothelial cells treated with the indicated concentrations of cycloastragenol in the Formulation eye drop formulation. FIG. 4A shows the effect of cycloastragenol concentration on telomerase activity in the corneal endothelial cell line HCEC-B4G12 treated for 30 hours. Untreated (NC) and placebo (Formulation eye drop formulation with 1% DMSO carrier but no cycloastragenol). Data were mean±standard deviation obtained 3 independent experiments, each repeating thrice. FIG. 4B is a line plot of the data shown in FIG. 4A.

FIGS. 5A-5B show time dependent changes to relative cell density (CD) in corneal layer endothelial cells of rabbits treated with Formulation eye drop formulation. FIG. 5A shows the effect of cycloastragenol in the Formulation eye drop formulation on cell density at the end of months 0, 1, 3 and 6. Untreated (negative control), placebo (eye drop Formulation with DMSO carrier but no cycloastragenol) are compared with groups B, C and D containing cycloastragenol at 25, 100 and 500 ng/mL. Results shown are normalized to the 0-month data. FIG. 5B is a line plot of the data shown in FIG. 5A.

FIGS. 6A-6B show time dependent changes to relative cell density (CD) in corneal layer endothelial cells of rabbits treated with Formulation eye drop formulation. FIG. 6A shows the effect of cycloastragenol in the Formulation eye drop formulation on cell density at the end of months 0, 1, 3 and 6. Untreated (negative control), placebo (Formulation eye drop formulation with DMSO carrier but no cycloastragenol) are compared with groups B, C and D containing cycloastragenol at 25, 100 and 500 ng/mL. Results shown are normalized to the negative control group. FIG. 6B is a line plot of the data shown in FIG. 6A.

FIGS. 7A-7B show time dependent changes to relative cell density (CD) in corneal layer endothelial cells of rabbits treated with Formulation eye drop formulation using the placebo for normalization. FIG. 7A shows the effect of cycloastragenol in the Formulation eye drop formulation on cell density at the end of months 0, 1, 3 and 6. Untreated (negative control), placebo (Formulation eye drop formulation with DMSO carrier but no cycloastragenol) are compared with groups B, C and D containing cycloastragenol at 25, 100 and 500 ng/mL. FIG. 7B is a line plot of the data shown in FIG. 7A.

FIGS. 8A-8E are representative histology sections showing Mallory-Azan (left panel) and Hematoxylin-Eosin (H&E, right panel) staining of the corneal layer in the various treatment groups described in FIG. 7. FIG. 8A shows Mallory-Azan and H&E staining for negative control group. FIG. 8B shows Mallory-Azan and H&E staining for placebo group.

FIG. 8C shows Mallory-Azan and H&E staining for Group B (25 ng/mL). FIG. 8D shows Mallory-Azan and H&E staining for Group C (100 ng/mL). FIG. 8E shows Mallory-Azan and H&E staining for Group D (500 ng/mL).

FIG. 9 shows the effect of an eye drop formulation containing cycloastragenol on telomere length in the corneal endothelial cells of treated rabbits.

FIG. 10 is a histogram showing the distribution of telomere lengths in a representative sample. Bars represent the relative frequency for every particular fluorescence intensity normalized (X axis). The 20th percentile (red bars) indicates the particular length below which 20% of the telomeres have been observed. The median (MTL) and average (ATL) telomere length are also indicated in the histogram. This histogram also allows for the analysis of telomere length variability.

FIGS. 11A-11E show representative images used for telomere length analysis in corneal endothelial cells from control and treated rabbits. FIG. 11A is representative image used for telomere length analysis in corneal endothelial cells in rabbits treated with Group A. FIG. 11B is representative image used for telomere length analysis in corneal endothelial cells in rabbits treated with Group B. FIG. 11C is representative image used for telomere length analysis in corneal endothelial cells in rabbits treated with Group C. FIG. 11D is representative image used for telomere length analysis in corneal endothelial cells in rabbits treated with Group D. FIG. 11E is representative image used for telomere length analysis in corneal endothelial cells in rabbits treated with Group N.

### DETAILED DESCRIPTION OF THE INVENTION

As used herein, the following terms and phrases shall have the meanings set forth below. Unless defined otherwise, all technical and scientific terms used herein have the same meaning as commonly understood to one of ordinary skill in the art.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. The terms “comprise” and “comprising” are used in the inclusive, open sense, meaning that additional elements may be included.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “ophthalmic formulation” or “formulation” are interchangeable and includes eye drops formulation which comprises an indicated concentration of a telomerase activator as active agent. A “placebo” or “placebo formulation” is a formulation without the active agent.

As used herein, the term “subject” refers to any recipient of the ophthalmic formulation whether it contains an active agent or is a placebo. The subject is a mammal, preferably, a human.

As used herein, the term “condition” or “eye condition” are interchangeable and refers to any pathophysiological disorder or disease or change in vision or visual acuity of the eye occurring as a result of aging.

In one embodiment of the present invention there is provided an ophthalmic formulation comprising in an isotonic saline solution a telomerase activator; an antimicrobial agent; a chelator; and at least one excipient.

In this embodiment the telomerase activator may be cycloastragenol. Also in this embodiment the cycloastragenol concentration in the isotonic saline solution is about 3 ng/ml to about 500 ng/ml. In one aspect of this embodiment the cycloastragenol concentration in the isotonic saline solution is about 25 ng/ml. In another aspect the cycloastragenol concentration in the isotonic saline solution is about 100 ng/ml. In yet another aspect the cycloastragenol concentration in the isotonic saline solution is about 500 ng/ml.

Also in this embodiment the antimicrobial agent may be benzalkonium chloride at a concentration in the isotonic saline solution of about 0.005% (w/v) to about 0.02% (w/v). In a representative example the antimicrobial agent is benzalkonium chloride at a concentration in the isotonic saline solution of 0.01% (w/v).

In addition in this embodiment the chelator may be ethylenediaminetetraacetic acid, disodium salt at a concentration in the isotonic saline solution of about 0.005% (w/v) to about 0.02% (w/v). In a representative example the chelator is ethylenediaminetetraacetic acid, disodium salt at a concentration in the isotonic saline solution of about 0.01% (w/v).

Further in this embodiment the excipient is hydroxypropyl methyl cellulose, polyvinylpyrrolidone, ethanol, or dimethyl sulfoxide or a combination thereof. Further still the excipient concentration in the isotonic saline solution may be about 0.05% (w/v) to about 0.9% (w/v). In one aspect of this further embodiment the excipient concentration in the isotonic saline solution is about 0.05% (v/v) to about 1% (v/v). In another aspect the excipient is hydroxypropyl methyl cellulose at a concentration in the isotonic saline solution of about 0.125% (w/v).

In another embodiment of the present invention there is provided an eye drops formulation, comprising cycloastragenol at a concentration of about 3 ng/ml to about 500 ng/ml in an isotonic saline solution containing an antimicrobial agent, a chelator and an excipient.

In this embodiment the cycloastragenol concentration in the isotonic saline solution may be selected from the group consisting of 25 ng/ml, 100 ng/ml and 500 ng/ml.

Also in this embodiment the antimicrobial agent contained in the isotonic saline solution may be benzalkonium chloride at a concentration of about 0.01% (w/v).

In addition the chelator contained in the isotonic saline solution may be ethylenediaminetetraacetic acid, disodium salt at a concentration in the isotonic saline solution of about 0.01% (w/v).

Further the excipient contained in the isotonic saline solution is at least one of hydroxypropyl methyl cellulose, polyvinylpyrrolidone, ethanol, or dimethyl sulfoxide at a concentration of about 0.05% (w/v) to about 0.9% (w/v) or is about 0.05% (v/v) to about 1% (v/v). In a representative example the excipient contained in the isotonic saline solution is hydroxypropyl methyl cellulose at a concentration of about 0.125% (w/v).

In yet another embodiment of the present invention there is provided a method for reducing the incidence of an age-related eye condition in an subject comprising administering to one or both eyes of the subject an amount of the ophthalmic formulation as described supra effective to increase an activity of telomerase in the corneal tissue thereby reducing the incidence of age-related eye condition.

In this embodiment the ophthalmic formulation may be administered topically. Also in the embodiment the ophthalmic formulation may be administered at least once daily over a period of about one month to about twelve months. In addition the age-related eye condition may be macular degeneration, cataract, presbyopia, or glaucoma, or a combination thereof.

In yet another embodiment of the present invention there is provided a method for increasing viability of cells in the corneal tissue of a subject, comprising contacting both eyes of the subject with an amount of the ophthalmic formulation as described supra.

In this embodiment the contacting step may comprise topically contacting one or both of the eyes with the ophthalmic formulation. Also in this embodiment the eye may be contacted with the ophthalmic formulation at least once daily. In addition the subject may have an age-related eye condition. Particularly, the age-related eye condition may be macular degeneration, cataract, presbyopia, or glaucoma, or a combination thereof.

Provided herein are ophthalmic formulations effective for increasing, telomerase activity, increasing telomere length and increasing the viability of cells in corneal tissue. The ophthalmic formulations generally are formulated in a saline solution, preferably, an isotonic saline solution and contain a telomerase activator as the active compound, an antimicrobial agent, a chelator, and at least one excipient, for example, any suitable physiologically acceptable excipient. The ophthalmic formulations may be eye drops.

The telomerase activator may be any compound that is known in the art to prevent loss of telomeres from the ends of the chromosomes or to promote addition of telomere sequences to the ends of the chromosomes. Alternatively, the telomerase activator may a combination of these compounds in the same or varying concentrations. In a non-limiting example, the telomerase activator is cycloastragenol at a concentration of about of about 3 ng/ml to about 500 ng/ml in the isotonic saline solution.

The antimicrobial agent protects the formulation from bacterial and/or fungal growth. The antimicrobial agent or a combination of antimicrobial agents may be used at any suitable concentration that would prevent growth of microbes and one of ordinary skill in this art would be readily able to discern the desired concentration that would prevent microbial growth. A non-limiting example of an antimicrobial agent is benzalkonium chloride at a concentration of about 0.005% (w/v) to about 0.02% (w/v), for example, about 0.01% (w/v), in the isotonic saline solution. Alternatively, the ophthalmic formulation containing the telomerase activator may be combined with other ophthalmic formulations in clinical use for the treatment of eye diseases including, but not limited to, formulations for glaucoma and age-related macular degeneration (AMD).

The chelator increases shelf life by improving stability of the formulation and may be used at any suitable concentration. In non-limiting examples the chelator is an ethylenediamine salt, a ethylenediaminetetraacetic acid disodium salt and an ethyleneglycoltetraacetic acid salt. Suitable concentrations for the chelator are about 0.005% (w/v) to about 0.02% (w/v), for example, about 0.01% (w/v), in the isotonic saline. The chelator may be disodium ethylenediaminetetraacetic acid disodium salt.

Each excipient is “physiologically acceptable” in the sense of not being injurious to biological cells and the subject, of being compatible with the other components in the formulation, and of not limiting the efficacy of the telomerase activator. The excipient may comprise a single compound or a combination of compounds in varying proportions, each ranging in concentration from about 0.05% (w/v) to about 0.9% (w/v) or from about 0.05% (v/v) to about 1% (v/v). In non-limiting examples, the excipient is at least one of hydroxypropyl methyl cellulose, polyvinylpyrrolidone, ethanol, or dimethyl sulfoxide. The excipient may be hydroxypropyl methyl cellulose at a concentration of about 0.125% (w/v). Alternatively, the excipients may be hydroxypropyl methyl cellulose and dimethylsulfoxide.

Also provided are methods that reduce the incidence of an age-related eye condition and/or increase the viability of cells in corneal tissue that utilize the ophthalmic formulations or eye drops described herein. The formulations or eye drops are administered to one or both eyes of a subject or the eyes are contacted with the formulations or eye drops. The age-related conditions of the eyes is any clinical ophthalmic condition that is caused due to decreased viability and/or reduced telomere length in cells of the corneal tissue or that occurs naturally as one ages. These conditions may include, but are not limited to, macular degeneration, cataract, presbyopia and glaucoma or a combination thereof.

Routes for administering or contacting the eye(s) are well-known routes applicable to a particular condition or to reduce or delay the incidence or onset of a particular condition and/or useful during an ophthalmic surgical procedure. For example, useful routes are, but not limited to, topical, intraocular, intravitreal, or system. One of skill in this art is well able to determine the most appropriate delivery method in view of the condition of the eyes.

Generally, one skilled in this art is well-able to determine the dose and dosage routine of the formulations depending on the health of the subject and the condition affecting the eyes. The formulations may be administered or the eye(s) contacted with the formulations or eye drops once, twice, or more times daily depending on the type and/or progression of the condition affecting the eye(s). This routine may occur for about one month to about 12 months. Alternatively, the formulation may be utilized until the condition has improved or until the condition has resolved in the subject.

The following examples are given for the purpose of illustrating various embodiments of the invention and are not meant to limit the present invention in any fashion.

### Example 1

### Preparation of Eye Drop Formulation

### I. Chemicals

1. Hydroxypropyl methyl cellulose (HPMC), Pharma Grade, H3785 SIGMA-ALDRICH

### 2. Benzalkonium Chloride, Pharma Grade, 12063 SIGMA-ALDRICH

3. EDTA Disodium Salt 2-hydrate, Pharma Grade, 141669 APPLICHEM

### 4. Dimethyl Sulfoxide (DMSO), Pharma Grade, 191954 APPLICHEM

5. 0.9% (w/v) Isotonic sodium chloride solution for infusion
6. Cycloastragenol (Active Compound), Bionorm Natural Products Co. Ltd.

### Devices

### 1. Esco Labculture® Class 2 Type 2a Biosafety Cabinet

2. ORION 5-STAR pH/ISE/Conductivity/DO Benchtop Multiparameter Meter

### 3. BROOKFIELD DV-E Viscometer

### Formulation Eye Drop Content

1. (Hydroxypropyl) methyl cellulose (HPMC): 0.125% (w/v), 1.25 mg/mL
2. Benzalkonium Chloride: 0.01% (w/v), 0.1 mg/mL
3. EDTA Disodium Salt 2-hydrate: 0.01% (w/v), 0.1 mg/mL
4. 0.9% Isotonic Sodium Chloride Solution for infusion
5. Active compound active dose range: Cycloastragenol, 3 to 500 ng/mL

### Preparation of the Hydroxypropyl Methyl Cellulose (HPMC) Solution

HPMC solution was made using the ‘hot-melt’ method. 20 mL of isotonic sodium chloride (0.9% w/v) is heated to 80° C. and added to 125 mg of HPMC. The mixture was allowed to stand for 1 hour at room temperature. 40 mL of isotonic saline was added to this mixture to obtain a viscous solution of HPMC.

### Preparation of Benzalkonium Chloride Solution

10 mg of Benzalkonium Chloride was dissolved in 10 mL saline solution and added to the HPMC solution.

### Preparation of EDTA Disodium Solution

10 mg of EDTA Disodium salt was dissolved in 10 mL saline solution and added to the HPMC/Benzalkonium Chloride solution.

### Preparation of Formulation Eye Drop

A 1 mg/mL main stock solution of the active compound, cycloastragenol was prepared in DMSO. The stock solution was diluted with DMSO to obtain three intermediate stock solutions having cycloastragenol at 0.5 mg/mL, 0.1 mg/mL and 0.025 mg/mL. To make Formulation eye drops having cycloastragenol at 25, 100 or 500 ng/mL, 100 μL of the appropriate intermediate stock solution was mixed with the 80 ml of HPMC/Benzalkonium Chloride/EDTA disodium solution prepared above, and the volume made to 100 mL using isotonic sodium chloride solution (0.9% w/v). The pH of the resulting solution was adjusted between 7.2 and 7.4 using 0.1 N sodium hydroxide. The solution was aseptically filtered through 0.22 μm filters in a laminar flow cabin and stored in sterile amber colored bottles.

### Preparation of Placebo Eye Drop

For preparing the placebo eye drops, the same procedure was followed as described above except that cycloastragenol was omitted to give eye drops having just the carrier, DMSO.

Formulation and placebo eye drops were preserved by using cold chain technology and stored in a special refrigerator at 2°-4° C.

### Viscosity Measurements

The viscosity of the eye drop solution was measured using a Brookfield DV-E viscometer (Table 1).

### II. Stability of the Formulation Eye Drop Formulation

The formulation was tested for stability by microbiological testing and pH and viscosity measurements.

TABLE 1
Viscosity measurements of Formulation eye drops

| Cycloastragenol | | | |
|---|---|---|---|
| concentration | | Speed | |
| in formulation | Torque | (revolutions | Viscosity |
| eye drops | (%) | per minute) | (mPas) |
| 500 ng/mL | 10.8 | 100 | 6.96 |
| 100 ng/mL | 10.2 | 100 | 7.18 |
| 25 ng/mL | 10.6 | 100 | 7.24 |
| 0 ng/mL (Placebo) | 10.8 | 100 | 6.82 |

### Microbial Testing

Group N—negative control

Group A—placebo

Group B—Active compound (cycloastrogenol), 25 ng/mL

Group C—Active compound (cycloastrogenol), 100 ng/mL

Group D—Active compound (cycloastrogenol), 500 ng/mL

The five groups of drugs above were incubated in the stability chamber at +4° C., +24° C. and +40° C. for 0, 3 and 6 months. At the end of the incubations, each group was subjected to stability test, sterility test, viscosity change test and pH change test. In addition, 6 months stability test groups were chromatographed by HPLC to verify the absence of degraded cycloastragenol. Tables 2 and 3 show representative integration results and peak analysis for the HPLC chromatogram of cycloastragenol after 6 months.

TABLE 2
Integration results from the HPLC chromatogram

| | Retention | | | Relative | Relative | |
|---|---|---|---|---|---|---|
| | Time | Area | Height | Area | Height | |
| No. | (min) | (mAU*min) | (mAU) | (%) | (%) | Amount |
| 1 | 1 | 249.386 | 135.355 | 6.41 | 1.91 | n.a. |
| 2 | 2.023 | 22.711 | 255.215 | 0.58 | 3.59 | n.a. |
| 3 | 2.09 | 24.996 | 266.542 | 0.64 | 3.75 | n.a. |
| 4 | 2.377 | 0.188 | 3.329 | 0 | 0.05 | n.a. |
| 5 | 2.52 | 45.477 | 1287.375 | 1.17 | 18.12 | n.a. |
| 6 | 2.57 | 139.271 | 2034.883 | 3.58 | 28.64 | n.a. |
| 7 | 3.08 | 2562.213 | 2410.722 | 65.9 | 33.93 | n.a. |
| 8 | 3.41 | 0.199 | 4.962 | 0.01 | 0.07 | n.a. |
| 9 | 4.127 | 10.677 | 41.953 | 0.27 | 0.59 | n.a. |
| 10 | 6.893 | 29.114 | 26.836 | 0.75 | 0.38 | n.a. |
| 11 | 8.697 | 133.511 | 41.635 | 3.43 | 0.59 | n.a. |
| 12 | 11.987 | 294.308 | 154.01 | 7.57 | 2.17 | n.a. |
| 13 | 14.17 | 50.054 | 114.164 | 1.29 | 1.61 | n.a. |
| 14 | 14.557 | 144.334 | 109.463 | 3.71 | 1.54 | n.a. |
| 15 | 15.967 | 27.767 | 94.767 | 0.71 | 1.33 | n.a. |
| 16 | 16.983 | 146.45 | 78.325 | 3.77 | 1.1 | n.a. |
| 17 | 18.763 | 3.369 | 18.833 | 0.09 | 0.27 | n.a. |
| 18 | 18.84 | 2.417 | 16.157 | 0.06 | 0.23 | n.a. |
| 19 | 19.043 | 1.352 | 9.295 | 0.03 | 0.13 | n.a. |
| 20 | 19.7 | 0.383 | 1.4 | 0.01 | 0.02 | n.a. |
| Total | — | 3888.177 | 7105.222 | 100 | 100 | — |

TABLE 3
Peak analysis of the HPLC chromatogram

| | Retention | Width | | | | |
|---|---|---|---|---|---|---|
| | Time | 50% | | Resolution | Asymmetry | Plates |
| No. | (min) | (min) | Type | (EP) | (EP) | (EP) |
| 1 | 1 | n.a. | BM | n.a. | n.a. | n.a. |
| 2 | 2.023 | n.a. | M | n.a. | n.a. | n.a. |
| 3 | 2.09 | n.a. | MB | n.a. | n.a. | n.a. |
| 4 | 2.377 | n.a. | BM | n.a. | n.a. | n.a. |
| 5 | 2.52 | n.a. | M | n.a. | n.a. | n.a. |
| 6 | 2.57 | n.a. | M | n.a. | n.a. | n.a. |
| 7 | 3.08 | 0.176 | M | n.a. | n.a. | 1692 |
| 8 | 3.41 | n.a. | Rd | n.a. | n.a. | n.a. |
| 9 | 4.127 | n.a. | Rd | n.a. | n.a. | n.a. |
| 10 | 6.893 | n.a. | Rd | n.a. | n.a. | n.a. |
| 11 | 8.697 | n.a. | Rd | n.a. | n.a. | n.a. |
| 12 | 11.987 | n.a. | M | n.a. | n.a. | n.a. |
| 13 | 14.17 | n.a. | M | n.a. | n.a. | n.a. |
| 14 | 14.557 | n.a. | M | n.a. | n.a. | n.a. |
| 15 | 15.967 | n.a. | M | n.a. | n.a. | n.a. |
| 16 | 16.983 | n.a. | M | n.a. | n.a. | n.a. |
| 17 | 18.763 | n.a. | M | n.a. | n.a. | n.a. |
| 18 | 18.84 | n.a. | M | n.a. | n.a. | n.a. |
| 19 | 19.043 | n.a. | MB | n.a. | n.a. | n.a. |
| 20 | 19.7 | 0.292 | BMB | n.a. | 1 | 25188 |

### Sterility Tests

Sterility tests were performed as described (1, 2). Sterility test results showed that there was no microbial growth in the 6 months stability test group at +4° C. and it was safe for use. Thus, the stability test results indicate that shelf life of the eye drop formulation was found to be 6 months at +4° C.

TABLE 4
Stability assessments from viscosity and pH of formulation eye drops

| | Cycloastrgenol | | Viscosity | | |
|---|---|---|---|---|---|
| | concentration | | | Speed | |
| Time, | in formulation | | Torque | (revolutions | Viscosity |
| temperature | eye drops | pH | (%) | per minute) | (mPas) |
| 0 days | 500 ng/mL | 7.38 | 12.1 | 100 | 7.26 |
| | 100 ng/mL | 7.35 | 12.4 | 100 | 7.44 |
| | 25 ng/mL | 7.35 | 12.3 | 100 | 7.38 |
| | 0 ng/mL | 7.34 | 12.2 | 100 | 7.32 |
| | (Placebo) | | | | |
| 30 days, 4° C. | 500 ng/mL | 6.65 | 11.5 | 100 | 6.90 |
| | 100 ng/mL | 6.71 | 11.6 | 100 | 6.96 |
| | 25 ng/mL | 6.71 | 11.2 | 100 | 6.72 |
| | 0 ng/mL | 6.64 | 11.1 | 100 | 6.66 |
| | (Placebo) | | | | |
| 30 days, 25° C. | 500 ng/mL | 6.60 | 11.4 | 100 | 6.84 |
| | 100 ng/mL | 6.77 | 11.2 | 100 | 6.72 |
| | 25 ng/mL | 6.90 | 11.3 | 100 | 6.78 |
| | 0 ng/mL | 6.68 | 11.0 | 100 | 6.60 |
| | (Placebo) | | | | |
| 30 days, 40° C. | 500 ng/mL | 6.64 | 11.6 | 100 | 6.96 |
| | 100 ng/mL | 6.76 | 11.1 | 100 | 6.66 |
| | 25 ng/mL | 6.72 | 11.4 | 100 | 6.84 |
| | 0 ng/mL | | | | |
| | (Placebo) | 7.24 | 10.8 | 100 | 6.48 |

### Viscosity and pH Measurements

The formulations were kept in a time and temperature-controlled stability cabinet at +25° C. (Room Temperature), +40° C. and +4° C. for 0 and 30 days followed by viscosity and pH measurements. Table 4 shows the results of pH and viscosity measurements after exposing the formulation for 30 days at 4° C., 25° C. and 40° C.

A decrease in the pH of the eye drops to 6.6 was observed after 30 days. On the other hand, the decrease in viscosity observed is negligible and would not be expected to affect activity.

### Example 2

### Cell Culture Studies

All experiments were designed and carried out in a randomized, double blind manner using placebos.

### Cytotoxicity Analysis

Corneal endothelial cell HCEC-B4G12 was used to determine in vitro effects of Formulation eye drop formulation on cell viability. Cytotoxicity was analyzed using the 2,5-Diphenyltetrazolium Bromide (MTT) assay at cycloastragenol concentrations ranging from 3.1 ng/mL and 100 μg/mL.

### Telomerase Activity

Effect of Formulation eye drop formulation on telomerase activity was evaluated in cornea endothelial cell HCEC-B4G12 using commercially available TeloTAGGG™ Telomerase PCR ELISA assay. The assay was performed 30 h after incubating the cells with the formulation containing 3.1 ng/mL to 100 μg/mL cycloastragenol.

### Results

### Cytotoxicity Analysis

The corneal cell line HCEC-B4G12 (DSMZ, ACC 647) was used to examine the dose and time dependent in vitro effect of the Formulation eye drop on cell viability of corneal endothelial cells as well as its cytotoxic activity. Subculture, incubation, harvest and storage transactions were made in compliance with the protocol that DSMZ suggests for this cell line. Formulation eye drop containing cycloastragenol was prepared fresh for each treatment as described above. The cells were treated with the formulation to get final cycloastragenol concentrations between 3.1 ng/mL and 100 μg/mL. After 48 hours, the effect of the medicine on cell viability and/or cytotoxic activity was determined using the 3-(4,5-Dimethylthiazol-2-yl)-2,5-Diphenyltetrazolium Bromide (MTT) test. Data are mean of three independent experiments each repeated thrice.

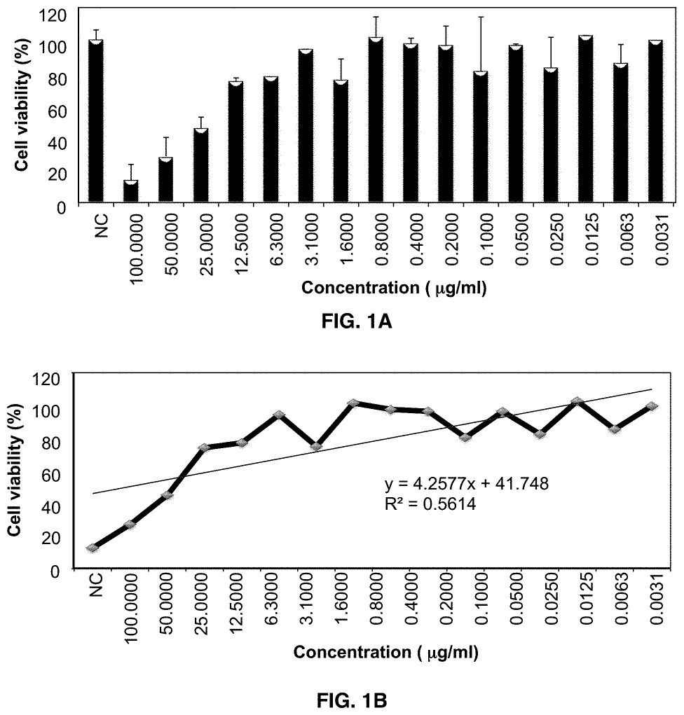

In general, it was observed that cell viability decreased as the cycloastragenol concentration was increased. Assuming the cell viability as 100% in the negative control (1% DMSO) and when the other values are normalized based on this value, cell viability values compared to the concentrations determined are shown in Table 5. IC₅₀ value was calculated as 40.2 μg/mL (Table 5; FIG. 1A-1C).

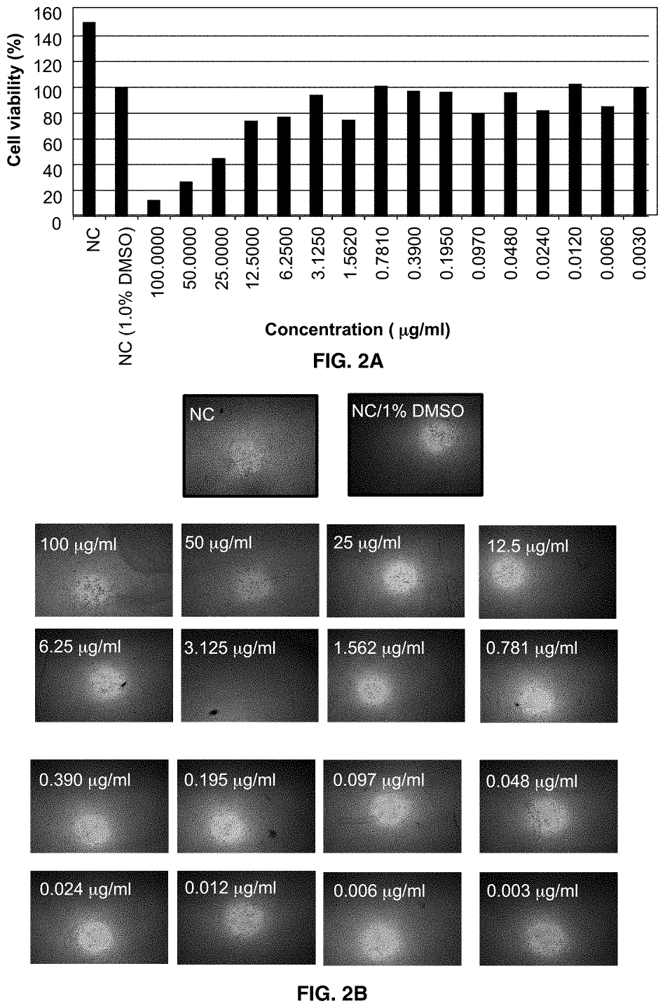

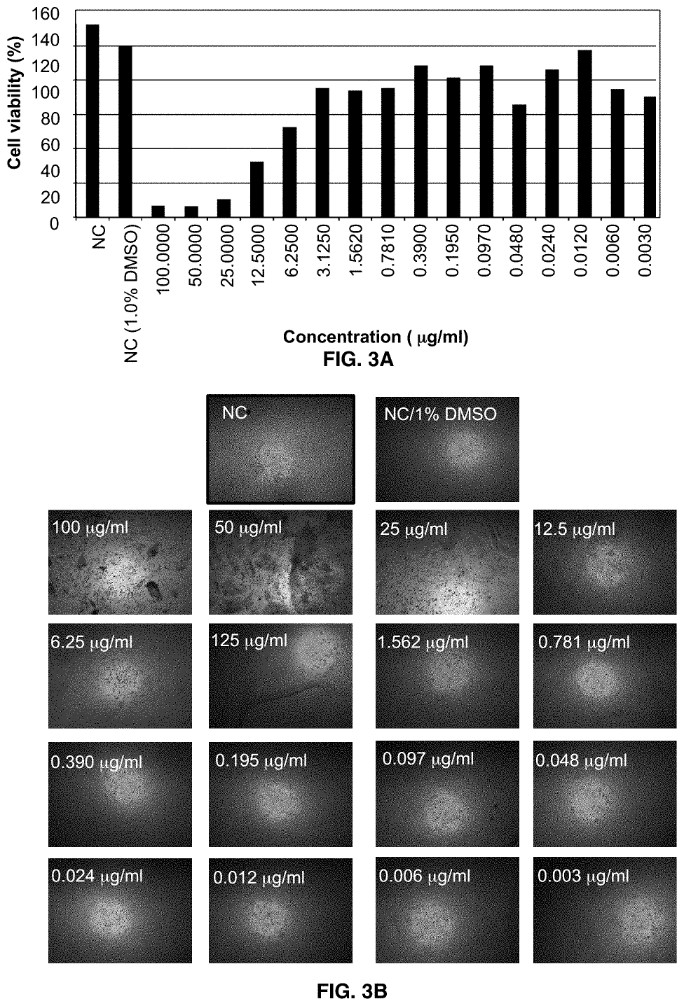

FIGS. 2A and 3A show cell viability data for corneal endothelial cells incubated with Formulation eye drops in 1% and 0.2% DMSO respectively, and FIGS. 2B and 3B show representative photomicrographs of the control and treated corneal endothelial cells.

TABLE 5
Cell Viability Values

| Concentration (μg/mL) | % Cell Viability ± Std.Dev. |
|---|---|
| 0* | 100 |
| 100 | 12.79 ± 6.30 |
| 50 | 27.02 ± 10.72 |
| 25 | 44.99 ± 13.09 |
| 12.5 | 74.06 ± 7.58 |
| 6.3 | 77.11 ± 2.62 |
| 3.1 | 94.18 ± 0.02 |
| 1.6 | 74.95 ± 0.07 |
| 0.8 | 101.41 ± 13.42 |
| 0.4 | 97.49 ± 12.87 |
| 0.2 | 96.4 ± 3.66 |
| 0.1 | 80.38 ± 12.22 |
| 0.05 | 96.25 ± 33.80 |
| 0.025 | 82.41 ± 1.30 |
| 0.0125 | 102.63 ± 19.31 |
| 0.00625 | 85.39 ± 0.23 |
| 0.00313 | 99.57 ± 11.90 |

Cells were treated with the active compound (Formulation) in an eye drop. Data shown were obtained after 48 hours. Results are mean of 3 independent experiments, each repeated thrice.

### TeloTAGGG™ Telomerase Activity

Corneal cell line HCEC-B4G12 (Deutsche Sammlung von Mikroorganismen and Zellkulturen (DSMZ) ACC-647) has been used in the study of researching the dose dependent in vitro effect of the Formulation eye drop on telomerase enzyme activity. Subculture, incubation, harvest and storage transactions were made in compliance with the protocol that DSMZ suggests for this cell line.

The Formulation eye drop containing cycloastragenol was prepared fresh for each treatment as described above. Cells were treated with Formulation eye drop formulation to get final cycloastragenol concentrations from 3.7 μg/mL to 10 ng/mL. The effect of Formulation on telomerase enzyme activity was determined using a TeloTAGGG™ Telomerase PCR ELISA assay (Roche, CAS Number 11854666910,). TeloTAGGG™ Telomerase PCR ELISA assay was performed as described by manufacturer (Sigma-Aldrich).

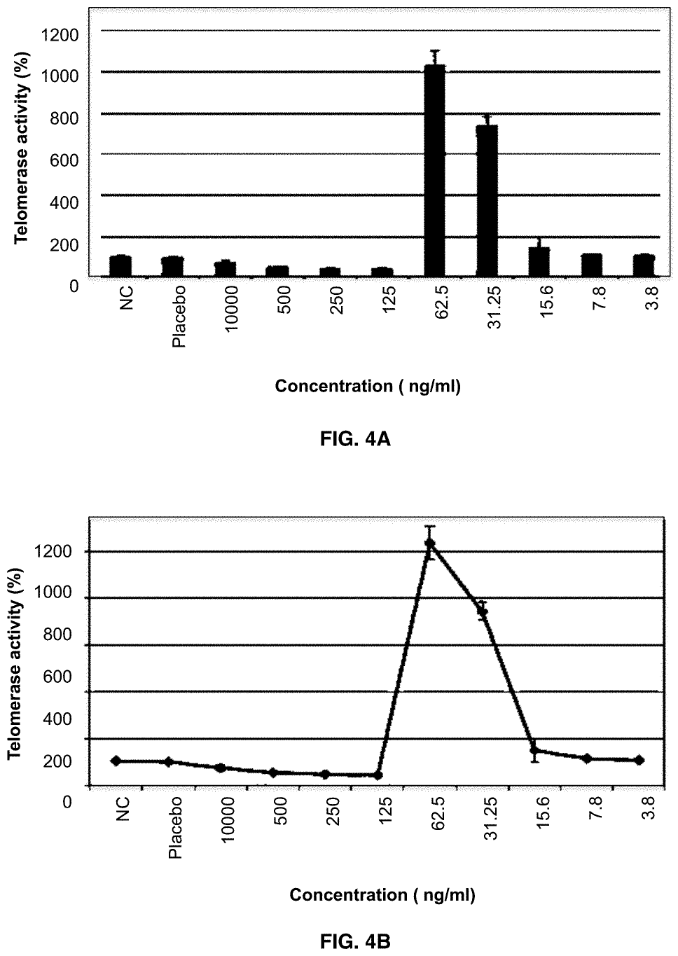

High Formulation concentrations in the range of 125-1000 ng/mL were found to be lower than negative control and lower Formulation concentrations in the range of 3.8-62.5 ng/mL were found to have higher telomerase activity than negative control. The highest activity of telomerase was obtained at 62.5 ng/mL Formulation. The telomerase activity obtained at this concentration was 10.35 times higher than the negative control (Table 6, FIGS. 4A and 4B). Cycloastragenol showed very high telomerase enzyme activity of corneal cells (HCEC-B4G12) in the concentration range of 62.5-31.25 ng/mL (1034.92-741.35%, respectively). This activity is approximately 7-10 times higher than the negative control. The activity decreases dramatically at the application concentrations below and above these concentrations (Table 4, FIGS. 4A and 4B). Assuming the telomerase enzyme activity is 100% in the negative control containing 1% DMSO (placebo) and when the other values are normalized based on this value, telomerase enzyme activity values compared to the concentrations are determined as shown in Table 6 and FIGS. 4A and 4B.

### Example 3

### Animal Studies

All experiments were designed and carried out in a randomized, double blind manner using placebos. Rabbits were housed for one week prior to the study, fed on standard laboratory food and allowed free access to water in a room at standard temperature and humidity conditions in a 12-h light/12-h dark cycle.

TABLE 6
Telomerase Enzyme Activity

| Treatment | % Telomerase Activity ± SD |
|---|---|
| NC | 103.8 ± 4.7 |
| Placebo | 100.00 |
| Concentration (ng/ML) | |
| 10000 | 74.0 ± 10.45 |
| 500 | 54.38 ± 2.78 |
| 250 | 46.28 ± 2.02 |
| 125 | 43.14 ± 0.24 |
| 62.5 | 1034.92 ± 69.41 |
| 31.25 | 741.35 ± 38.49 |
| 15.6 | 148.94 ± 49.54 |
| 7.8 | 114.42 ± 2.79 |
| 3.8 | 108 ± 5.27 |

The corneal cell line HCEC-B4G12 was treated with the active compound (Formulation) in an eye drop. At a concentration range from 3.8 to 10000 ng/mL for 30 hours. Data were result of 3 independent experiments, each repeated thrice.

### Animal Model and Surgical Procedure:

Rabbits were randomly divided and distributed into five groups:
Group N—negative control
Group A—placebo
Group B—Active compound (cycloastrogenol), 25 ng/mL
Group C—Active compound (cycloastrogenol), 100 ng/mL
Group D—Active compound (cycloastrogenol), 500 ng/mL
Four rabbits (eight eyes) were used per group. This design provided a statistical significance p 0.001.
Counting Corneal Cell number:

Each rabbit was treated with 0.1 mL of eye drop formulation twice a day for six months. The formulation was prepared in 15-day volumes. Any excess was discarded, and a fresh batch prepared for the next 15-days. Effects of the eye drop on corneal endothelium as well as cell numbers and shapes were observed by means of specular microscopy (NIDEK-CEM 530, Japan) at the end of months 0, 1, 3 and 6. Rabbits were euthanatized by an intravenous injection of Tanax (0.3 mL/kg). Vitreous (0.2 mL) was aspirated with a 25-gauge needle attached to a 5-mL disposable syringe, driven at 3.0 mm from the limbus, and guided toward the center of the eyeball, taking care to avoid bleeding when the needle was introduced (vitreous samples were stored at −80° C. until the analysis). Eye tissue was fixed with 4% formaldehyde and embedded in paraffin for histopathological assessment. The individual values for three replicates were determined and the mean values were reported.

### Statistical Analysis and Methods:

The statistical significance of the differences between the concentrations of active compound following administration of the drops and the corresponding controls was tested at each time point by a one-way analysis of variance (ANOVA) with the pairwise multiple comparison procedures (Student-Newman-Keuls Method) for multiple comparison (SigmaStat program; Jandel Scientific, Version 1.0) and expressed as means±standard error (SEM) of three independent experiments. All analyses were carried out using GraphPad Prism 5.0 (GraphPad Software, San Diego, Calif., USA). Differences were considered to be significant at a level of P 0.001.

### Histopathology

Eye tissue was fixed with 4% formaldehyde and embedded in paraffin for the histopathological assessment and processed according to routine light microscope tissue processing methods. 5 μm tissue sections stained with Hematoxylin-Eosin (H&E) were examined and photographed by Leica image analyzing system (Leica, Germany). All specimens were evaluated individually by three histologists who were blinded to the drug type and the treatment times. Histopathology was also performed to evaluate the corneal opacity, neovascularization, inflammatory cell density, vessel size and edema. Moreover, lens structure, scleral, choroidal and macular changes were analyzed by histopathology.

### Telomere Length Analysis

Samples were received in paraffin. Different methods were used in order to obtain the best results during the dewaxing process. After dewaxing, the tissue samples were processed according to Life Length's cell disaggregation protocol to generate single-cell suspensions appropriate for the analysis. This was followed with a filter step to eliminate collagen residues in the preparation.

### Results

### Corneal Endothelial Cells Counting

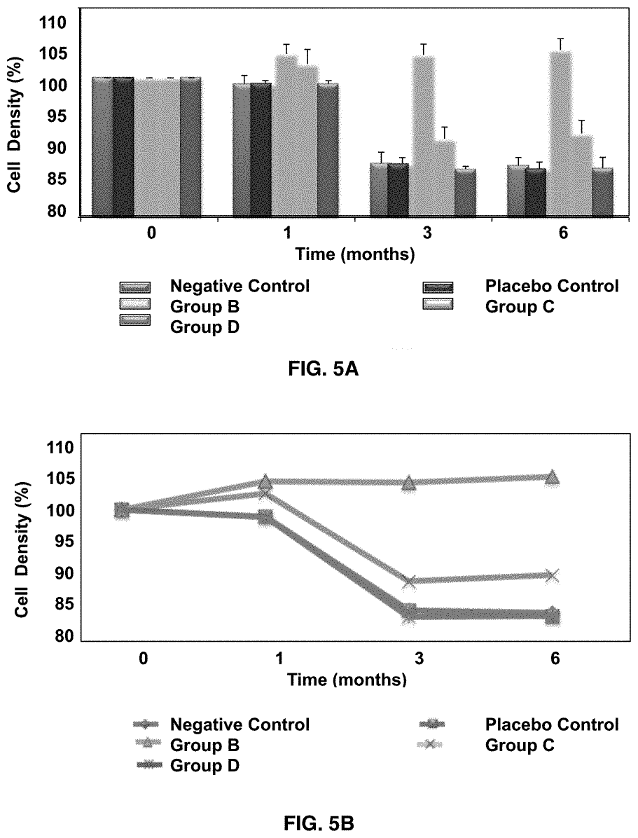

Cornea layers from the eyes of untreated (negative control, Group N), carrier treated (placebo, Group A) and cycloastragenol-treated (Groups B, C and D) rabbits were analyzed by specular microscopy at the end of 0, 1, 3 and 6 months. Time dependent changes in the density of corneal endothelial cells (CD parameter) were compared and these are presented in Tables 5-8 and in FIGS. 5-7. The data shows that relative change in corneal endothelial cell density in the negative control group was 99.12 (±1.22) at the end of month 1, 87.74 (±1.61) at the end of month 3 and 87.45 (±1.14) at the end of month 6 (normalized to month 0), which corresponds to a 1%, 12% and 13% reduction in cell density respectively. At the end of month 6, it was observed that the endothelial cells in this group degenerated by 13% as a result of apoptosis and/or autophagy (y=−4.9028x+105.84; R²=0.8372) (Table 7, FIGS. 5A and 5B). In the placebo group, corneal endothelial cell density was found to be 99.17 (±0.43) at the end of month 1, 87.72 (±0.85) at the end of month 3 and 87.00 (±0.92) at the end of month 6, similar to the negative control group (y=−5.0455x+106.09; R²=0.8479) (Table 7, 10; FIGS. 5A and 5B). Thus, time dependent changes in the density (CD parameter) of endothelial cells in cornea layers of the eyes from the negative control and placebo experimental group have very similar values and they change parallel to each other suggesting that the carrier does not have any negative effect on cell viability.

TABLE 7
Relative changes to cell density following Formulation treatment

| | CD ± SEM | | | |
|---|---|---|---|---|
| Time | | Time | | Time |
| (months) | 0 | (months) | 0 | (months) |
| Group N | 100.00 ± 0.00 | Group N | 100.00 ± 0.00 | Group N |
| (Negative | | (Negative | | (Negative |
| Control) | | Control) | | Control) |
| Group A | 100.00 ± 0.00 | Group A | 100.00 ± 0.00 | Group A |
| (Placebo) | | (Placebo) | | (Placebo) |
| Group B | 100.00 ± 0.00 | Group B | 100.00 ± 0.00 | Group B |
| Group C | 100.00 ± 0.00 | Group C | 100.00 ± 0.00 | Group C |
| Group D | 100.00 ± 0.00 | Group D | 100.00 ± 0.00 | Group D |

Relative changes to cell density (CD parameter) of cornea layer endothelial cells in negative control (no treatment), placebo (carrier alone) and Formulation treatment groups B, C and D containing cycloastragenol at 25, 100 and 500 ng/mL respectively. Results are normalized to month 0 and shown at the end of months 0, 1, 3 and 6.

For the treatment group B (25 ng/mL), the corneal endothelial cell density was 103.44 (±1.34) for month 1, 103.29 (±1.53) for month 3 and 104.00 (±1.61) for month 6, which corresponds to a 3%, 3% and 4% change in cell density respectively. Thus, apoptosis and/or autophagy (natural cell death) in group B slows down and/or stops as evidenced by an increase in density by 4% (y=1.848x+99.72; R²=0.7109), (Table 6, FIGS. 5A and 5B).

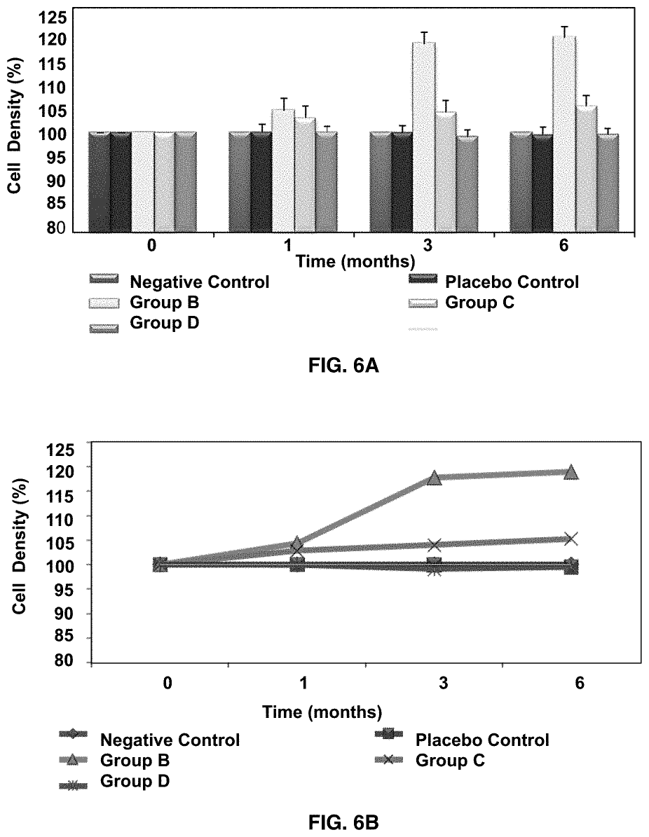

Comparison of relative changes to density of corneal layer endothelial cells in the negative control group and treatment group B shows a higher cell density for group B at about 4% in month 1, 18% in month 3 and 19% in month 6 (y=7,0134x+92,716; R²=0,9082), (Table 8, FIGS. 6A and 6B). At the end of the month 6, endothelial cells from group B was 19% higher in density compared to the negative control group. Consequently, compared to the negative control group, the effect of 25 ng/mL cycloastragenol on slowing down and stopping apoptosis and/or autophagy as a consequence of natural cell aging was determined to be 100%, which corresponds to a 0% degeneration rate (Table 8, FIGS. 6A and 6B).

TABLE 8
Relative changes to cell density following Formulation treatment

| Time | CD ± SEM | | | |
|---|---|---|---|---|
| (months) | 0 | 1 | 3 | 6 |
| Group N | 100.00 ± 0.00 | 100.00 ± 0.00 | 100.00 ± 0.00 | 100.00 ± 0.00 |
| (Negative | | | | |
| Control) | | | | |
| Group A | 100.00 ± 0.00 | 100.05 ± 1.52 | 99.97 ± 1.33 | 99.49 ± 1.61 |
| (Placebo) | | | | |
| Group B | 100.00 ± 0.00 | 104.36 ± 1.11 | 117.72 ± 1.38 | 118.93 ± 1.17 |
| Group C | 100.00 ± 0.00 | 102.86 ± 2.49 | 104.02 ± 2.35 | 105.24 ± 2.19 |
| Group D | 100.00 ± 0.00 | 99.99 ± 1.16 | 99.09 ± 1.47 | 99.54 ± 1.25 |

Relative changes to cell density (CD parameter) of cornea layer endothelial cells in negative control (no treatment), placebo (carrier alone) and Formulation treatment groups B, C and D containing cycloastragenol at 25, 100 and 500 ng/mL respectively. Results are normalized to Group N and shown at the end of months 0, 1, 3 and 6.

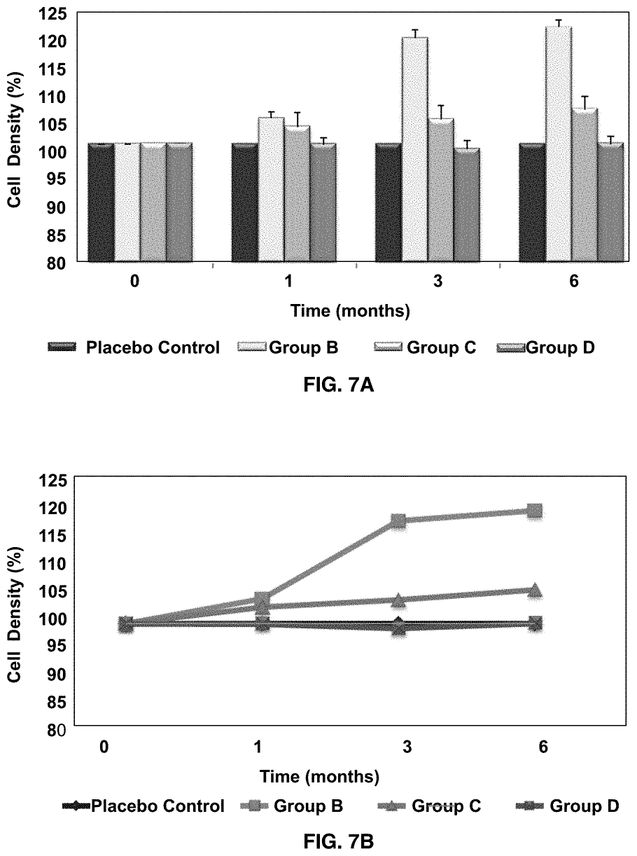

Comparison of relative changes to density of corneal layer endothelial cells in the placebo group and treatment group B shows a higher cell density for group B at about 4% in month 1, 18% in month 3 and 20% in month 6 (y=7,2068x+92,382; R²=0,9179) (Table 9, FIGS. 7A and 7B). At the end of the month 6, endothelial cells from group B was 20% higher in density compared to the placebo group. Consequently, compared to the placebo group, the effect of 25 ng/mL cycloastragenol on slowing down and stopping apoptosis and/or autophagy as a consequence of natural cell ageing was determined to be 100%, which corresponds to a 0% degeneration rate (Table 9, FIGS. 7A and 7B). Table 10 summarizes the results of cell density for the various treatment groups.

A significance analysis comparison between treatment groups using T-Student analysis is shown. Key: p>0.1 not significant; * p<0.01; ** p<0.0001.

### Histopathological Analysis

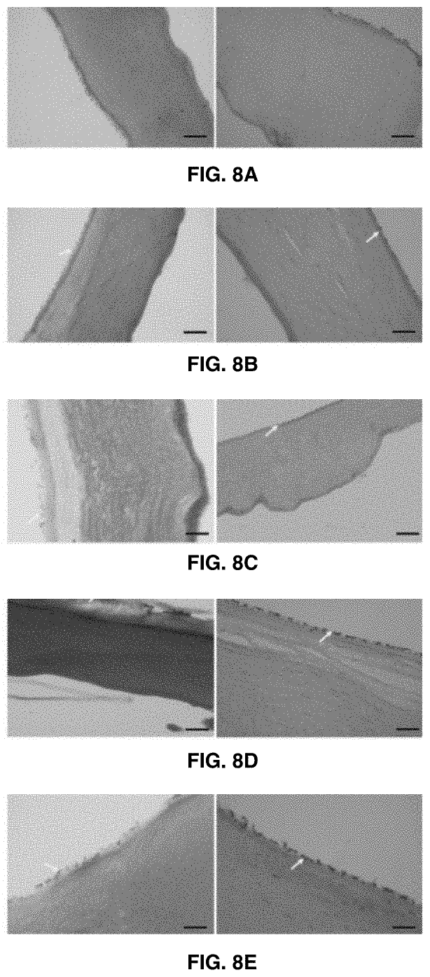

Group N (Negative control): Corneal endothelium was observed to form a layer of single-layer, hexagonal cells located on the descement membrane. Corneal endothelial cells were found to have a regular hexagonal alignment. Cell configurations in this group were found to be compatible with maternal corneal endothelium. No loss was found in the number of cells (FIG. 8A).

TABLE 9
Relative changes to cell density following Formulation treatment

| Time | CD ± SEM | | | |
|---|---|---|---|---|
| (months) | 0 | 1 | 3 | 6 |
| Group A | 100.00 ± 0.00 | 100.00 ± 0.00 | 100.00 ± 0.00 | 100.00 ± 0.00 |
| (Placebo) | | | | |
| Group B | 100.00 ± 0.00 | 104.31 ± 1.11 | 117.75 ± 1.38 | 119.54 ± 1.17 |
| Group C | 100.00 ± 0.00 | 102.81 ± 2.49 | 104.05 ± 2.35 | 105.79 ± 2.19 |
| Group D | 100.00 ± 0.00 | 99.94 ± 1.16 | 99.12 ± 1.47 | 100.05 ± 1.25 |

Relative changes to cell density (CD parameter) of cornea layer endothelial cells in negative control (no treatment), placebo (carrier alone) and Formulation treatment groups B, C and D containing cycloastragenol at 25, 100 and 500 ng/mL respectively. Results are shown at the end of months 0, 1, 3 and 6.

TABLE 10
Cell Density (CD) of cornea layer endothelial cells in eyes from treated animals

| | CD ± SEM | | | |
|---|---|---|---|---|
| Time | 0 | 1 | 3 | 6 |
| Group N (Negative) | 2715.83 ± 235.02 | 2681.75 ± 143.43 | 2401.00 ± 87.97 | 2340.80 ± 57.68 |
| Group A | 2903.0 ± 170.75 | 2870.5 ± 60.84 | 2438.50 ± 104.55 | 2319.75 ± 104.05 |
| (Placebo Control) | | | | |
| Group B | 2531.00 ± 211.52 | 2612.38 ± 129.09 | 2604.50 ± 78.17** | 2545.88 ± 45.42* |
| Group C | 2689.88 ± 91.28 | 2741.13 ± 141.29 | 2453.88 ± 125.12 | 2472.38 ± 37.90** |
| Group D | 2875.13 ± 251.01 | 2835.38 ± 177.28 | 2498.50 ± 331.49 | 2488.88 ± 241.53 |

*p ≤ 0.001, **p ≤ 0.01.

Group A (Placebo): Corneal endothelium was found to consist of single-layer cells placed on the descement membrane. Where there are cell losses in place, histopathological degenerative effects were observed in terms of cell nuclei and their shape. It is concluded that this is compatible with the surgical intervention. It was observed that the nuclei of some cells increased in size and lost their natural properties (FIG. 8B).

Group B: Corneal endothelium consisted of single-layer cells placed on the descement membrane. Compared to group A, cell losses were observed in very few areas. Cell nuclei were found to be in proper configuration compared to Group A. Group D and Group C compared the number of cell losses in the group where the cell configuration and number of the group was found to be the closest to Group NC (FIG. 8C).

Group C: Corneal endothelium was also seen in this group that formed a layer of single-layer; hexagonal cells located on the descement membrane. Group B and Group D compared with the number of cell losses seen in this group in the group where the configuration and number of cells were found to be close to Group NC (FIG. 8D).

Group D: The corneal endothelium was found to form a single-layer, hexagonal close-layered layer on the descement membrane. Although cell configurations were found to be compatible with mature corneal endothelium in the samples belonging to this group, compared to Group B and Group C, the more hypertrophic and place cell losses of the cells were found to be different from these two groups (FIG. 8E).

Measurement of Telomerase Length in Corneal Endothelial Cells from Treated Animals

Six-month-old male New Zealand rabbits were treated (100 μl/eye, 6 months) with negative control (Group N), placebo (Group A) or the formulation of the present invention containing 25, 100, 500 ng/mL cycloastragenol (Groups B, C, D).

TABLE 11
Telomere length results from TAT analysis

| | | Median | 20^{th} | | |
|---|---|---|---|---|---|
| | | Length | Percentile | | |
| | | (Base | Length | Telomeres | CV |
| Formulation | Group | Pairs) | (Base Pairs) | <3 Kbp (%) | % |
| Negative | GROUP N | 10,310 | 6,123 | 5.2 | 0.7 |
| Control | | | | | |
| 0 ng/mL | GROUP A | 10,274 | 6,362 | 5.4 | 0.6 |
| (Placebo) | | | | | |
| 25 ng/mL | GROUP B | 11,431 | 6,968 | 3.7 | 1.1 |
| 100 ng/mL | GROUP C | 11,543 | 7,374 | 3.1 | 1.2 |
| 500 ng/mL | GROUP D | 11,552 | 7,113 | 3.6 | 2 |

Median telomere length and 20th percentile median telomere length is shown for each treatment group. The percentage of short telomeres (<3 Kbp) are also shown. Data are mean from five experiments.

Samples of the eye tissue were fixed and received in paraffin. After dewaxing, the tissue samples were processed according to Life Length's cell disaggregation protocol comprising mechanical and enzymatic steps to generate single-cell suspensions appropriate for the analysis. The sample is filtered to remove collagen residues. Telomere lengths were assessed using Life Length's.

TABLE 12
T-Student analysis of telomere length

| | | | 20^{th} | | | |
|---|---|---|---|---|---|---|
| | Median | | Percentile | | | |
| | Length | | Length | | Telomeres <3 | |
| Comparison | p Value | Significance | p Value | Significance | Kbpp Value | Significance |
| Group A vs | 0.00005 | **** | 0.03 | * | 0.12 | not |
| Group B | | | | | | significant |
| Group A vs | 0.0003 | *** | 0.002 | ** | 0.007 | ** |
| Group C | | | | | | |
| Group A vs | 0.0001 | *** | 0.012 | * | 0.02 | * |
| Group D | | | | | | |
| Group A vs | 1 | not | 0.5 | not | 0.6 | not |
| Group N | | significant | | significant | | significant |
| Group B vs | 0.7 | not | 0.1 | not | 0.3 | not |
| Group C | | significant | | significant | | significant |
| Group B vs | 0.3 | not | 0.6 | not | 0.7 | not |
| Group D | | significant | | significant | | significant |
| Group B vs | 0.002 | ** | 0.02 | * | 0.08 | not |
| Group N | | | | | | significant |
| Group C vs | 0.8 | not | 0.3 | not | 0.2 | not |
| Group D | | significant | | significant | | significant |
| Group C vs | 0.002 | ** | 0.003 | ** | 0.02 | * |
| Group N | | | | | | |
| Group D vs | 0.003 | ** | 0.011 | * | 0.04 | * |
| Group N | | | | | | |

A significance analysis comparison between treatment groups using T-Student analysis is shown.
Key: p > 0.1 not significant;
* p < 0.05;
** p < 0.01;
*** p < 0.001;
**** p < 0.0001.ian

### TAT Analysis Protocol.

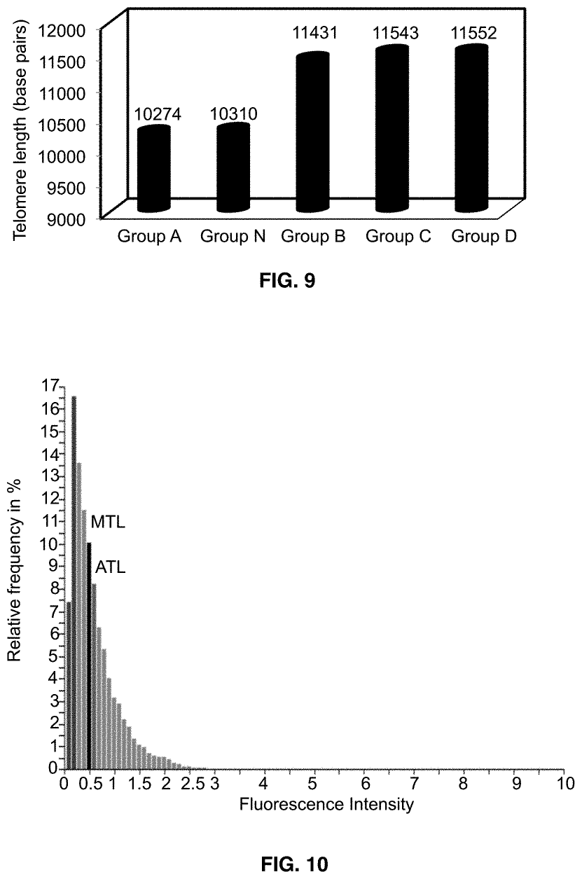

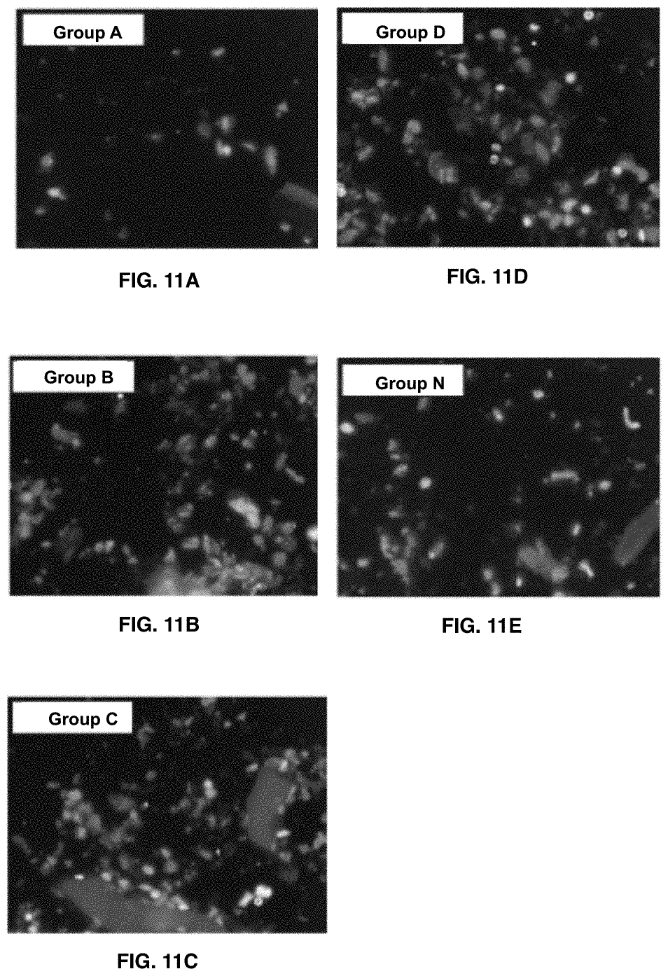

Table 11 shows the median telomere length and 20th percentile median telomere length (both in base pairs—bp) for each sample, as well as the percentage of short telomeres defined as the percentage of the telomeres with a length below 3 Kbp (<3 Kbp). All measurements were performed in quintuplicate. The T-Student analysis for significance is summarized in Table 12. FIG. 9 is a graphical presentation of the telomere length analysis. FIG. 10 shows a representative histogram showing distribution of telomere lengths. FIGS. 11A-11E show representative images from the treatment groups used for telomere length analysis in corneal endothelial cells.

The following references are cited herein.
1. Uddin, M. et al. Journal of Advances in Medical and Pharmaceutical, 14(2): 2017, 1-17.
2. Evaluation and Recommendation of Pharmacopoeial Texts for Use in the ICH Regions, International Conference on Harmonisation of Technical Requirements for Registration Of Pharmaceuticals For Human Use, Annex 8 Sterility Test, November 2008.
3. Sandie, T., Journal of GXP Compliance, 18 (3), 2014, 1-5.
4. World Health Organization, Test for Sterility, Document QAS/11.413 FINAL March 2012, 1-9.
5. U.S. Pharmacopeia, Sterility Tests, Chapter 71, 2017.

What is claimed is:

1. An ophthalmic formulation comprising:
in an isotonic saline solution:
a telomerase activator;
an antimicrobial agent;
a chelator; and
at least one excipient.

2. The ophthalmic formulation of claim 1, wherein the telomerase activator is cycloastragenol.

3. The ophthalmic formulation of claim 2, wherein the cycloastragenol concentration in the isotonic saline solution is about 3 ng/ml to about 500 ng/ml.

4. The ophthalmic formulation of claim 1, wherein the antimicrobial agent is benzalkonium chloride at a concentration in the isotonic saline solution of about 0.005% (w/v) to about 0.02% (w/v).

5. The ophthalmic formulation of claim 1, wherein the chelator is ethylenediaminetetraacetic acid disodium salt at a concentration in the isotonic saline solution of about 0.005% (w/v) to about 0.02% (w/v).

6. The ophthalmic formulation of claim 1, wherein the excipient is hydroxypropyl methyl cellulose, polyvinylpyrrolidone, ethanol, or dimethyl sulfoxide or a combination thereof.

7. The ophthalmic formulation of claim 7, wherein the excipient concentration in the isotonic saline solution is about 0.05% (w/v) to about 0.9% (w/v).

8. The ophthalmic formulation of claim 7, wherein the excipient concentration in the isotonic saline solution is about 0.05% (v/v) to about 1% (v/v).

9. A method for reducing the incidence of an age-related eye condition in an subject comprising:
administering to one or both eyes of the subject an amount of the ophthalmic formulation of claim 1 effective to increase an activity of telomerase in the corneal tissue thereby reducing the incidence of age-related eye condition.

10. The method of claim 9, wherein the ophthalmic formulation is administered topically.

11. The method of claim 9, wherein the ophthalmic formulation is administered at least once daily over a period of about one month to about twelve months.

12. The method of claim 9, wherein the age-related eye condition is macular degeneration, cataract, presbyopia, or glaucoma, or a combination thereof.

13. A method for increasing viability of cells in the corneal tissue of a subject, comprising:
contacting both eyes of the subject with an amount of the ophthalmic formulation of claim 1.

14. The method of claim 13, wherein the contacting step comprises topically contacting both eyes with the ophthalmic formulation.

15. The method of claim 13, wherein the subject has an age-related eye condition.

16. The method of claim 13, wherein the age-related eye condition is macular degeneration, cataract, presbyopia, or glaucoma, or a combination thereof.

17. An eye drops formulation, comprising:
cycloastragenol at a concentration of about 3 ng/ml to about 500 ng/ml in an isotonic saline solution containing an antimicrobial agent, a chelator and an excipient.

18. The eye drops formulation of claim 17, wherein the cycloastragenol concentration in the isotonic saline solution is selected from the group consisting of 25 ng/ml, 100 ng/ml and 500 ng/ml.

19. The eye drops formulation of claim 17, wherein the antimicrobial agent contained in the isotonic saline solution is benzalkonium chloride at a concentration of about 0.01% (w/v).

20. The eye drops formulation of claim 17, wherein the chelator contained in the isotonic saline solution is ethylenediaminetetraacetic acid disodium salt at a concentration in the isotonic saline solution of about 0.01% (w/v).

21. The eye drops formulation of claim 17, wherein the excipient contained in the isotonic saline solution is at least one of hydroxypropyl methyl cellulose, polyvinylpyrrolidone, ethanol, or dimethyl sulfoxide at a concentration of about 0.05% (w/v) to about 0.9% (w/v) or is about 0.05% (v/v) to about 1% (v/v).

22. The eye drops formulation of claim 21, wherein the excipient contained in the isotonic saline solution is hydroxypropyl methyl cellulose at a concentration of about 0.125% (w/v).
